(19) United States
(12) Patent Application Publication
(10) Pub. No.: US 2008/0249634 A1
(43) Pub. Date: Oct. 9, 2008

(54) METHOD FOR PRODUCING A HOLLOW PROFILE BASED ON A CROSS-LINKED, GELATINOUS MATERIAL AND IMPLANTS IN THE FORM OF HOLLOW PROFILES

(75) Inventor: Michael Ahlers, Eberbach (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 12/120,344

(22) Filed: May 14, 2008

Related U.S. Application Data
(63) Continuation of application No. PCT/EP2006/008948, filed on Sep. 14, 2006.

(30) Foreign Application Priority Data
Nov. 17, 2005 (DE) 10 2005 054 943.8

(51) Int. Cl. A61F 2/04; A61L 33/00

(52) U.S. Cl. 623/23.64; 427/2.25

(57) ABSTRACT

A method for producing a hollow profile comprising a cross-linked, gelatinous material, the hollow profile having a polygonal cross-section and comprising a wall, which surrounds a lumen, is provided, the method comprising:
a) preparing an aqueous solution of a gelatinous material;
b) partially cross-linking the gelatinous material in the solution;
c) applying the solution to the surface of a shaped element which defines the lumen; and
d) leaving the solution to dry at least partially on the shaped element, thus forming a hollow profile comprising the cross-linked, gelatinous material.

### CROSS-REFERENCE TO RELATED APPLICATIONS

This patent application is a continuation of PCT Application No. PCT/EP2006/008948, filed Sep. 14, 2006, which claims priority of German patent Application No. 10 2005 054 943.8, filed Nov. 17, 2005, which are each incorporated by reference.

### BACKGROUND OF THE INVENTION

The present invention relates to a method for producing a hollow profile based on a cross-linked, gelatinous material.

The invention also relates to implants in the form of hollow profiles.

Implants in the form of hollow profiles, i.e. in particular tubular implants, find use in diverse areas of medicine. An important field of use is in this regard insertion of implants of this kind in order to hold open the lumen of tubular organs or tissues and to prevent collapse of vessel walls. Implants of this kind are also called stents and are used for example in blood vessels, the intestines and the esophagus.

In other cases, hollow profiles are implanted in order to fulfil a drainage function, i.e. to conduct for example tissue fluid from an inflamed region.

The implants described above are in part made of a durable material, such as for example stainless steel, and must therefore be removed again, when they are no longer required to fulfil their function. In order to spare the patient this further intervention (in part operative), implants of resorbable biopolymers are therefore an important alternative, biopolymers such as for example gelatin, collagen or chitosan, which are broken down by the body after a certain time.

### SHORT SUMMARY OF THE INVENTION

It is an object of the present invention to put forward a method for production of resorbable hollow profiles which can be carried out in a simple manner and by means of which the properties of the profile produced may be controlled over an extensive range.

This object is met according to the invention by a method for producing a hollow profile based on a cross-linked, gelatinous material, the hollow profile having a polygonal cross-section and comprising a wall, which surrounds a lumen, the method comprising:
a) preparing an aqueous solution of a gelatinous material;
b) partially cross-linking the dissolved, gelatinous material;
c) applying the solution to the surface of a shaped element which defines the lumen; and
d) leaving the solution to dry at least partially on the shaped element, a hollow profile based on the cross-linked, gelatinous material being formed.

By a polygonal cross-section there is to be understood in the context of the present invention, every cross-section of the hollow profile which has a finite or infinite number of corners, i.e. in particular also oval, elliptical or round cross-sections. Hollow profiles with a round cross-section, i.e. cylindrical hollow profiles, represent, within the scope of the present invention, the simplest case and indeed the shape which is most commonly required.

### DETAILED DESCRIPTION OF THE INVENTION

Production according to the invention of the hollow profile using a shaped element which defines the lumen has clear advantages compared with other methods of manufacture. It can be realised with little technical complexity, the dimensions of the hollow profile and its cross-sectional shape being readily varied by the choice of shaped element and also being able to be specified with a high level of accuracy.

Extrusion of the hollow profile would be associated with significantly higher expense. In addition there is here the problem that in the case of use, according to the invention, of gelatin as starting material, the advantages of which will be further gone into below, extrusion can only be carried out under temperature conditions such as lead to partial thermal breakdown of the gelatin.

A further possibility for producing hollow profiles would be the rolling up of a flat material, such as for example a film. This inevitably leads however to non-homogeneous material properties for the hollow profile, at least along a seam; in addition, these methods cannot really be used for very small diameters of the hollow profile.

Application of the solution to the surface of the shaped element is effected in the case of a preferred embodiment of the invention by dipping the shaped element into the solution. In this way, a very uniform application of the solution to the shaped element is ensured, this leading to a hollow profile with a wall of substantially uniform thickness. After the hollow profile has been left to dry at least partially, it may be removed from the shaped element.

Alternatively, there is the possibility of spraying the solution of the gelatinous material onto the shaped element. However, in this case, it is not possible to achieve quite so uniform an application without special precautions.

The shaped element should preferably be formed from an inert material with a smooth surface, such as for example stainless steel. In order to facilitate the subsequent withdrawal of the hollow profile, the shaped element may be treated with a separating agent (for example wax) before application of the solution.

While the dimensions of the lumen, i.e. in particular the internal diameter of the hollow profile, may be determined by the shaped element defining the lumen, the thickness of the wall is dependent on the quantity of solution applied. This quantity can in turn be controlled primarily by the viscosity of the solution, in particular in the case when the shaped element is used in a dipping step.

The wall thickness of the hollow profile can be still further increased if steps c) and d) are repeated one or more times subsequent to step d). In particular, this relates to multiple dipping of the shaped element into the solution, a uniform coating of material being applied each time.

Now that application of the solution of the gelatinous material to the shaped element has first of all been described, the composition of the material and the cross-linking will now be gone into in detail in the following text, the cross-linking having to take place, according to the invention, before application of the solution to the shaped element.

For production of hollow profiles which are insoluble under physiological conditions but are resorbable, use according to the invention of gelatin is extremely advantageous, since this can be resorbed by the body without leaving any trace. In contrast to the material collagen, which is related to gelatin, gelatin of high purity and reproducible composition is available and is free from immunogenic telopeptides, which can cause defensive reactions by the body.

In order to assure optimal bio-compatibility in medical use of the hollow profile produced, the material preferably contains a gelatin with an especially low content of endotoxins. Endotoxins are metabolic products or fragments of microorganisms, which are present in animal raw material. The endotoxin content of gelatin is specified in International Units per gram (I.U./g) and is determined by the LAL test, the carrying out of which is described in the fourth edition of the European Pharmacopoeia (Ph. Eur. 4).

In order to keep the content of endotoxins as low as possible, it is advantageous for the microorganisms to be killed off as early as possible in the course of preparation of the gelatin. Furthermore, suitable standards of hygiene should be observed in the preparation process.

Accordingly, the endotoxin content of gelatin can be drastically reduced by specific measures during the preparation process. Among these measures, there belong primarily use of fresh raw materials (for example, pig skin) with storage time being avoided, meticulous cleaning of the entire production installation immediately before beginning preparation of the gelatin, and optionally replacement of ion exchangers and filter systems in the production installation.

The gelatin used within the scope of the present invention preferably has an endotoxin content of 1,200 I.U./g or less, still more preferably, 200 I.U./g or less. Optimally, the endotoxin content is 50 I.U./g or less, in each case determined in accordance with the LAL test. By comparison with this, many commercially available gelatins have endotoxin contents of more than 20,000 I.U./g.

The gelatinous material used to produce the hollow profile is preferably formed to a preponderant extent from gelatin. This includes in particular gelatin fractions of 60% by weight or more, preferably 75% by weight or more. As well as gelatin, the material may contain for example still further biopolymers such as for example alginates or hyaluronic acid, in order to match the property profile of the hollow profile more specifically to a particular application.

In a preferred embodiment of the invention, the gelatinous material additionally comprises a plasticizer. By virtue of an additive of this kind, the flexibility of the hollow profile produced is significantly increased in the dry state. This may be of advantage if, for example, a high bending elasticity is desired for the hollow body during implantation. Glycerin, oligoglycerins, oligoglycols and sorbite are for example suitable as plasticizers, glycerin being most preferred.

The desired flexibility of the hollow profile may be controlled by way of the amount of plasticizer. Preferably, the fraction of plasticizer in the material is 12 to 40% by weight. Especially advantageous for this are fractions of 16 to 25% by weight. The weight percentages specified relate here to the total mass of all of those constituents of the gelatinous material which are present both in the solution in accordance with step a) and also in the hollow profile produced.

In a further exemplary embodiment of the method according to the invention, in particular when no addition of plasticizer is effected, the material is formed substantially entirely from gelatin.

The concentration of gelatin in the aqueous solution in accordance with step a) is preferably 5 to 45% by weight, most preferably 10 to 30% by weight. Concentrations in this range are in particular suitable for the dipping of the shaped element into the solution.

According to the invention, the gelatinous material is partially cross-linked in accordance with step b), the gelatin itself preferably being cross-linked. Since gelatin is intrinsically water soluble, this cross-linking is necessary, in order to inhibit excessively rapid disintegration of the hollow profile and to ensure an adequate lifespan under physiological conditions.

In this regard, gelatin offers the additional advantage that the speed of resorption of the cross-linked material or the length of time up to complete resorption, may be adjusted over a wide range by choice of the degree of cross-linking.

A particular embodiment of the method according to the invention further comprises cross-linking (step e)) of the material comprised in the hollow profile. Preferably, in this further cross-linking, the gelatin in particular is cross-linked.

The advantage of a two-stage cross-linking of this kind consists basically in the ability to achieve a greater degree of cross-linking and as a result, a long time to break down. This cannot be realised to the same extent in a single-stage method by raising the concentration of the cross-linking agent, since by virtue of too strong a cross-linking of the dissolved material, the material is no longer workable and cannot be applied to the surface of the shaped element.

On the other hand, use of a non-cross-linked material and cross-linking of it exclusively after production of the hollow profile is also not suitable, since in this case, the boundary surfaces accessible from the outside are more strongly cross-linked than in the inner regions of the hollow profile, which is reflected in non-homogeneous breakdown behavior.

The second cross-linking in accordance with step e) may be carried out by the action of an aqueous solution of a cross-linking agent, but is preferably the action of a gaseous cross-linking agent.

The cross-linking may be carried out either chemically or enzymatically.

Preferred chemical cross-linking agents are aldehydes, dialdehydes, isocyanates, carbodiimides and alkyl dihalides. Especially preferred is formaldehyde, in particular for the second cross-linking in the gas phase, sterilization of the hollow profile being effected by this at the same time.

As enzymatic cross-linking agent, the enzyme transglutaminase is preferably used, which effects linking of the glutamine and lysine side chains of proteins, in particular also of gelatin.

The hollow profiles produced according to the invention may to an extent have remarkably long lifespans under physiological conditions, and it is possible to set these lifespans very specifically by the degree of cross-linking. The resorption stability under standard physiological conditions provides a measure of this. Thus hollow profiles may be produced which, under standard physiological conditions, remain stable for example for longer than a week, longer than two weeks, or longer than four weeks.

The concept of stability is to be understood to the effect that the hollow profile substantially retains its original shape both during storage in the dry state and also during the specified time period under standard physiological conditions (PBS buffer, pH 7.2, 37° C.) and only subsequently breaks down structurally to a substantial extent by hydrolytic action. The physiological conditions to which the hollow profiles are exposed during their use as implants, are primarily characterized by temperature, pH value and ion strength, and may be simulated in vitro by incubation under the standard conditions mentioned.

Surprisingly, hollow profiles may also be produced with a high degree of cross-linking, which by the addition of a plasticizer may nonetheless provide a relatively high flexibility, i.e. lifespan and flexibility may be controlled to a certain extent independently of one another.

In another embodiment of the method according to the invention, the hollow profile is stretched in the longitudinal direction subsequent to step d). Stretching of this kind may have a positive effect in a variety of respects. For one thing, the at least partial alignment of the gelatin molecules along a preferred direction leads to improved mechanical properties for the hollow profile, i.e. to raising tear strength and/or ultimate elongation, in particular in the longitudinal direction. This may be of great advantage in certain fields of use.

Furthermore, stretching facilitates the production of hollow profiles with a very small internal diameter, since longitudinal stretching leads to radial contraction of the hollow profile. Correspondingly small diameters, which could not readily be realised solely by means of a lumen-defining shaped element, are provided for example for hollow profiles for nerve guides for the peripheral nervous system.

The hollow profile may be stretched in an especially effective manner if the gelatinous material comprises a plasticizer.

Preferably, the hollow profile is brought into a thermoplastic state directly before stretching, by raising temperature and/or water content. This may for example be effected by the hollow profile being exposed to hot steam. Stretching of the hollow profile is advantageously carried out with a stretch ratio of 1.4 to 8, a stretch ratio of up to 4 being preferred.

In a further embodiment of the method according to the invention, the hollow profile is stored, before stretching, for up to four weeks, preferably at room temperature. In this way, the tear strength of the hollow profiles produced according to the invention can be further raised. Significant effects are here to be observed even after a storage time of only about three days, while from a storage time of about seven days, no further significant increase can as a rule be achieved.

If a second cross-linking (step e)) is carried out, this is preferably effected after stretching of the hollow profile, if this is carried out. This is therefore advantageous, because the molecules in the partially cross-linked material then still have sufficient freedom of movement and can therefore align themselves at least in part along a preferred direction and remain in this aligned state.

In a further embodiment of the method according to the invention, a reinforcing material is added to the solution produced by step a). In this way, reinforced hollow profiles with an increased mechanical strength can be produced. In particular, tearing of the hollow profile during surgical stitching may thereby be countered. The reinforcing materials should be physiologically compatible and at best also be resorbable.

Depending on the choice of reinforcing material, stability in respect of resorption mechanisms may be affected to a certain extent, along with the effect on mechanical properties. In particular, the resorption stability of the reinforcing materials may be selected independently of the constituents of the gelatinous material for the hollow profile.

The reinforcing materials show, even for fractions of 5% by weight, a marked improvement in the mechanical properties of the hollow profile. The fractions by weight here relate to the total dry mass in the solution in accordance with step a), i.e. all constituents of the solution with the exception of water.

Above 60% by weight, no further significant improvement can as a rule be achieved and/or the desired resorption properties or also the necessary flexibility of the hollow profile may be achieved only with difficulty.

The reinforcing materials may be selected from particulate and/or molecular reinforcing materials as well as mixtures of these.

In the case of particulate reinforcing materials, use of reinforcing fibers is particularly recommended. The fibers for this are selected preferably from polysaccharide and protein fibers, in particular collagen fibers, silk and cotton fibers, and from polylactide fibers and mixtures of any of the foregoing.

On the other hand, molecular reinforcing materials are also suitable in order to improve mechanical properties and, if desired, also to improve the resorption stability of the hollow profile.

Preferred molecular reinforcing materials are in particular polyactide polymers and their derivatives, cellulose derivatives, and chitosan and its derivatives. Molecular reinforcing materials may also be used as mixtures.

In is a further object of the invention to provide a resorbable implant in the form of a hollow profile, which can be used in very many ways and the properties of which can be adapted to particular requirements.

This object is met according to the invention by an implant in the form of a hollow profile, which is produced based on a cross-linked, gelatinous material, the hollow profile having a polygonal cross-section and comprising a wall which surrounds a lumen.

A multiplicity of possible modifications of a hollow body of this kind for the purpose of adapting its properties to the specific demands of an application have already been further described above in connection with the method according to the invention.

A preferred method for producing implants according to the invention is that described above.

Particular advantages which can be achieved by use of a cross-linked, gelatinous material have likewise already been described above in connection with the method according to the invention.

A preferred embodiment of the implant according to the invention relates to a stent for the esophagus. Stents of this kind are used in the event of a narrowing of the esophagus (stenosis of the esophagus), which can be congenital or acquired (e.g. by a chemical burn). In the case of congenital stenosis of the esophagus, which occurs primarily in children, there is present an abnormality in the development of the muscular system of the esophagus, so that this requires to be held open by means of a stent.

When a conventional stent for the esophagus is used, for example of stainless steel, this has to be removed after a specific time, which represents an additional strain on the patient and involves the risk of injury. By contrast, the resorbable stent according to the invention offers the advantage that it is broken down by the body after a specific prescribed time, when it is no longer needed or when a new stent has to be inserted. This latter is for example the case for children because of growth.

Because the degree of cross-linking of the gelatinous material can be specified, as described above, the time required for the stent to break down can be set as required for the particular treatment situation. Typically the time required for the stent to break down is one to two weeks.

Another advantageous field of use for implants according to the invention are stents for the intestines. These are used in particular in the case of surgical interventions in the section of intestine concerned, in order to prevent outflow of the content of the intestine in the event of leakage occurring. At the same time, for example, stitching (or an inflamed or diseased location) can be protected and healing can occur on the outer side of the stent. Here also the ability to specify the time required for the stent to break down proves to be especially advantageous.

A further possible field of use for the implant according to the invention is a stent for the trachea.

The stents described above preferably have an internal diameter of 5 to 30 mm, internal diameters from 8 to 20 mm being especially preferred for most instances of use.

Depending on the particular application, the gelatinous material, in the case of the implant according to the invention, may have a different degree of cross-linking in an inner region of the wall adjacent to the lumen, than in an outer region. In the case of stents for the intestines or the esophagus, it may for example be preferred for the degree of cross-linking to be somewhat less in the outer region. Because of this, the hollow profile may become gelled from the outside to a certain extent and thereby adhere to the esophagus or intestine. From the inside out, the stent remains by contrast as solid as possible and thereby provides good sliding qualities.

Along with the fields quoted above, implants according to the invention may also be used as stents for other organs or tissue, in particular for blood vessels, the ureter, the urethra, uterine tubes and the cystic duct.

In many of these cases, the implant or the stent may also serve as a temporary replacement for tissue and/or as a matrix for regeneration of tissue. In an application of this kind, the ends of a blood vessel which has been severed (or other tissue mentioned above) is introduced from both sides into the implant and thus connected. The hollow tube can then function as a matrix, which promotes growth and regeneration of tissue. At the same time, the implant provides a lateral barrier against foreign cells, which could possibly interfere with healing. The time required for the implant to break down may once again be adapted to the time required for regeneration of the tissue.

An especial field of use for guide structures according to the invention is nerve guides, which are used for regeneration of severed nerve fibers (axons). For this, the hollow profile has to have a very small internal diameter, preferably in the range from 800 to 1,200 μm.

Finally, another embodiment of the implant according to the invention relates to a drain. Drains of this kind are used inter alia in order to facilitate outflow of fluid (for example, blood or tissue fluid) from an inflamed or injured region. The fact that the diameter of implants according to the invention can be selected to be customised, as can the time required for breakdown, enables use as a drain in very many different regions of the body.

The implant according to the invention may also be used in particular as drainage for the eye. Drains of this kind, for which a very thin tubule is in question, are used to draw off intraocular fluid from the eyeball in the case of glaucoma.

The drain according to the invention for the eye preferably has an internal diameter of 50 to 200 μm.

Implants according to the invention which have small diameters, as required for example for nerve guides and drains for the eye, may be produced in particular by means of the method according to the invention, the hollow tubes being stretched as described above.

### SHORT DESCRIPTION OF THE SEVERAL VIEWS OF THE DRAWINGS

These and further advantages of the invention will be explained in more detail on the basis of the accompanying examples with reference to the figures. In particular:

FIG. 1: shows a photographic illustration of implants according to the invention; and

FIG. 2: is an image showing an implant according to the invention in cross-section, taken using an optical microscope.

### EXAMPLES

### Example 1

### Production of an Implant According to the Invention

This example relates to the production, by means of the method according to the invention, of an implant in the form of a hollow profile which has an internal diameter of 1 cm.

For this, 130 g of pig skin gelatin (Bloom strength 300 g) was initially dissolved at 60° C. in a mixture of 468 g of water and 52 g of glycerin as plasticizer and the solution was degassed by means of ultrasound. This corresponds to a plasticizer fraction in the material of about 29% by weight, based on the weight of gelatin and glycerin.

After addition of 6.5 g of an aqueous, 2.0% by weight formaldehyde solution (1000 ppm of cross-linker based on the gelatin), the solution was homogenized, again degassed, and the surface freed of foam. A stainless steel mandrel, serving as a shaped element and having a diameter of 1 cm, which had previously been sprayed with a separating wax, was dipped briefly into the solution to a length of about 10 cm. After the mandrel was withdrawn from the solution, it was rotated, so that the solution adhering formed as uniform a layer as possible.

After drying for approximately one day at 25° C. and a relative humidity of 30%, the formed hollow profile was removed from the mandrel. The implant produced in this way, which has an internal diameter of 1 cm, may be used for example as a stent for the esophagus.

In order to prolong the time for physiological degradation of the implant, the gelatin contained in it was submitted to a further cross-linking. For this, the implant was exposed, in a dessicator, for 17 hours to the equilibrium vapor pressure of an aqueous formaldehyde solution of 17% by weight, at room temperature.

An implant with a higher degree of cross-linking in the inner region may in this case also be obtained, for example by the formaldehyde vapor being conducted exclusively through the lumen of the hollow profile. Alternatively, different degrees of cross-linking may also be realised by the mandrel being dipped successively into solutions with different concentrations of cross-linking agent.

It will be understood that the properties of the implant described here may be modified in very many different ways, in that in particular the size and shape of the mandrel or shaped element, the fractions of gelatin, plasticizer and cross-linking agent in the solution, the number of immersion steps, and the intensity of the subsequent cross-linking may be adapted to the particular requirements.

### Example 2

### Production of Further Implants According to the Invention

This example concerns the production, by means of the method according to the invention, of implants in the form of hollow profiles which have small internal diameters of approximately 2,000 μm, 1,100 μm and 150 μm.

A solution of 100 g of pig skin gelatin (Bloom strength 300 g) in a mixture of 260 g of water and 40 g of glycerin as plasticizer was prepared as described in Example 1. This corresponds to a plasticizer fraction in the material of about 29% by weight, based on the weight of gelatin and glycerin.

After addition of 4 g of an aqueous, 2.0% by weight formaldehyde solution (800 ppm of cross-linker based on the gelatin), the solution was homogenized, again degassed, and the surface freed of foam. An array of stainless steel pins with a diameter of 2 mm, which had previously been sprayed with a separating wax, was dipped briefly into the solution to a length of about 3 cm. After the pins were withdrawn from the solution, they were held vertical, so that the solution adhering formed as uniform a layer as possible.

After drying for approximately one day at 25° C. and a relative humidity of 30%, it was possible to remove the formed hollow profiles (tubules) from the stainless steel pins. They have an internal diameter of 2,000 μm and a mean wall thickness of about 300 μm, this being established by optical microscope.

In order to produce hollow profiles with still smaller internal diameters, the tubules were stored for five days at 23° C. and a relative humidity of 45% and then stretched.

For stretching, the tubules were gripped at both ends and softened by the action of hot steam. In this thermoplastic condition, they were lengthened with a stretch ratio of about 1.4, fixed in this condition, and dried over a period of 16 hours at 23° C. and a relative humidity of 45%.

In order to prolong the time for physiological degradation of the tubules, they may, after stretching, be submitted to a further cross-linking in the gas phase, as described in Example 1. For this, the ends of the tubules may be closed, so that the cross-linking is effected only from the outside.

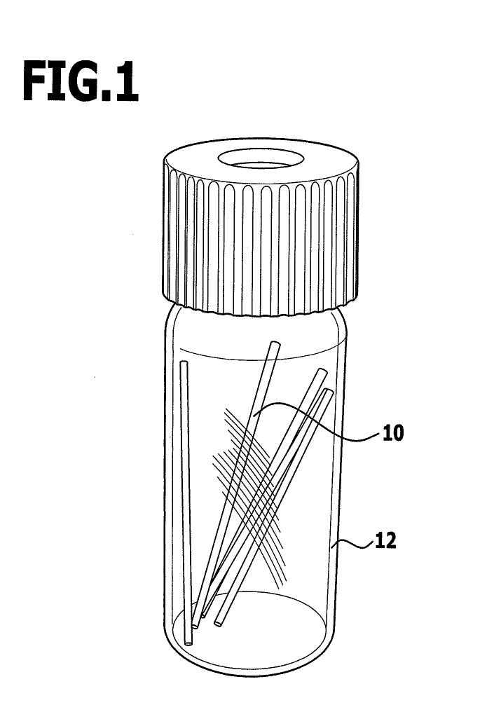

In FIG. 1, some tubules 10 produced in this way and having a length of about 3 cm, are shown in a glass container 12.

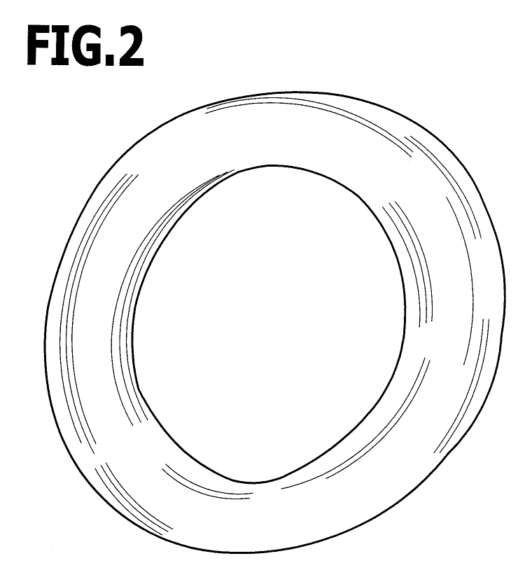

FIG. 2 shows an image taken using an optical microscope of the cross-section through one of the stretched tubules. This has an internal diameter of about 1,100 μm and a wall thickness of about 200 μm: both the cross-sectional shape and the wall thickness of the tubule are extremely consistent.

Implants with these dimensions may be especially well suited to use as nerve guides. Also, a strong cross-linking of the tubule starting from the outer side is advantageous for this usage, since in this way, the implant can become broken down starting from the inside as the nerve cells grow.

By raising the stretch ratio, implants according to the invention with an even smaller internal diameter may also be produced, which may be advantageous for other usages. In particular, it is possible by use of the method according to the invention, to produce extremely thin tubules having an internal diameter in the region of 150 μm, which may be used as a drain for the eye.

1. A method for producing a hollow profile comprising a cross-linked, gelatinous material, the hollow profile having a polygonal cross-section and comprising a wall, which surrounds a lumen, the method comprising:
a) preparing an aqueous solution of a gelatinous material;
b) partially cross-linking the gelatinous material in the solution;
c) applying the solution to the surface of a shaped element which defines the lumen; and
d) leaving the solution to dry at least partially on the shaped element, thus forming a hollow profile comprising the cross-linked, gelatinous material.

2. The method according to claim 1, the hollow profile having a round cross-section.

3. The method according to claim 1 wherein applying the solution to the surface of the shaped element comprises dipping the shaped element into the solution.

4. The method according to claim 1 c) and d) being repeated one or more times subsequent to d).

5. The method according to claim 1, wherein preparing the aqueous solution of the gelatinous material comprises obtaining a gelatin having an endotoxin content, as determined by the LAL test, of 1,200 I.U./g or less, and preparing the aqueous solution of the gelatinous material comprising said gelatin.

6. (canceled)

7. The method according to claim 1, wherein the aqueous solution is prepared from a gelatinous material comprising a plasticizer.

8. (canceled)

9. The method according to claim 7, the fraction of plasticizer in the gelatinous material being 12 to 40% by weight.

10. (canceled)

11. The method according to wherein the aqueous solution is prepared from a gelatinous material formed substantially entirely from gelatin.

12. The method according to claim 1, wherein the aqueous solution of the gelatinous material is prepared with a concentration of gelatin in the solution being 5 to 45% by weight.

13. (canceled)

14. The method according to claim 1, further comprising:
e) further cross-linking the gelatinous material comprised in the hollow profile.

15. (canceled)

16. The method according to claim 14, wherein further cross-linking of the gelatinous material comprises exposing the hollow profile to a cross-linking agent in the gas phase.

17-18. (canceled)

19. The method according to claim 1, comprising stretching the hollow profile in the longitudinal direction subsequent to d).

20. The method according to claim 19, comprising bringing the hollow profile into a thermoplastic state directly before stretching, by raising temperature and/or water content.

21. The method according to claim 19, comprising stretching the hollow profile with a stretch ratio of 1.4 to 8.

22. (canceled)

23. The method according to claim 19, comprising storing the hollow profile, before stretching, for up to four weeks.

24. The method according to claim 23, comprising storing the hollow profile, before stretching, for three to seven days.

25. The method according to claim 19, comprising further cross-linking (e)) of the gelatinous material after stretching of the hollow profile.

26. The method according to claim 1, comprising adding a reinforcing material to the solution produced according to a).

27. The method according to claim 26, the reinforcing material having a fraction of the dry mass of 5% by weight or more.

28. The method according to claim 26, the reinforcing material having a fraction of the dry mass of up to 60% by weight.

29. The method according to claim 26, the reinforcing material being selected from particulate and/or molecular reinforcing materials.

30. The method according to claim 29, the particulate reinforcing material comprising reinforcing fibers.

31. The method according to claim 30, the reinforcing fibers being selected from polysaccharide fibers and protein fibers, and from polyactide fibers and mixtures of any of the foregoing.

32. The method according to claim 30, the molecular reinforcing material being selected from polyactide polymers and their derivatives, cellulose derivatives, and chitosan and its derivatives.

33. An implant in the form of a hollow profile comprising a cross-linked, gelatinous material, the hollow profile having a polygonal cross-section and comprising a wall which surrounds a lumen.

34. (canceled)

35. The implant according to claim 33, the gelatinous material having a different degree of cross-linking in an inner region of the wall adjacent to the lumen than in an outer region.

36-38. (canceled)

39. The implant according to claim 36, the implant having an internal diameter of 5 to 30 mm.

40. The implant according to claim 36, the stent having an average wall thickness of 300 to 1,500 μm.

41-47. (canceled)

48. The implant according to claim 47, the implant having an internal diameter of 50 to 200 μm.

49. (canceled)

50. The method according to claim 5, wherein preparing the aqueous solution of the gelatinous material comprises obtaining a gelatin having an endotoxin content, as determined by the LAL test, of 200 I.U./g or less, and preparing the aqueous solution of the gelatinous material comprising said gelatin.

51. A method of treating a patient suffering from a stenosis of the esophagus, comprising implanting a stent into the esophagus, wherein the stent comprises an implant in the form of a hollow profile comprising a cross-linked, gelatinous material, the hollow profile having a polygonal cross-section and comprising a wall which surrounds a lumen.
